(19) United States
(12) Patent Application Publication
(10) Pub. No.: US 2014/0283851 A1
(43) Pub. Date: Sep. 25, 2014

(54) LAMINATED BANDAGE COMPRISING AN ACTIVATED CARBON CLOTH

(71) Applicant: Equi-Med Ag Limited, Durham (GB)

(72) Inventors: Kay Dowson, Durham (GB); Andrea Claire Smith, Durham (GB)

(21) Appl. No.: 14/352,476

(22) PCT Filed: Oct. 25, 2012
(86) PCT No.: PCT/GB2012/052660

§ 371 Date: Apr. 17, 2014

(30) Foreign Application Priority Data
Oct. 26, 2011 (GB) GB1118526.1

(51) Int. Cl. A61F 15/00

(52) U.S. Cl. CPC A61F 15/008
USPC 128/888

(57) ABSTRACT

The present invention relates to multi-layer products and methods utilising such products. Embodiments of the present invention have utility in the prevention and/or healing of wounds and skin disorders. More particularly, although not exclusively, the present invention relates to products and methods which have utility in the veterinary field. Embodiments of the present invention relate to products and methods which are for use in the prevention and/or healing of wounds and skin disorders in equines, namely, horses, ponies, donkeys and the like, particularly wounds and skin disorders which affect the limbs of such animals. Aspects of the present invention provide products and methods for supporting limb joints whilst allowing the joint to flex to enable movement.

### TECHNICAL FIELD

The present invention relates to multi-layer products and methods utilising such products. Embodiments of the present invention have utility in the prevention and/or healing of wounds and skin disorders. More particularly, although not exclusively, the present invention relates to products and methods which have utility in the veterinary field. Embodiments of the present invention relate to products and methods which are for use in the prevention and/or healing of wounds and skin disorders in equines, namely, horses, ponies, donkeys and the like, particularly wounds and skin disorders which affect the limbs of such animals. Aspects of the present invention provide products and methods for supporting limb joints whilst allowing the joint to flex to enable movement.

### BACKGROUND TO THE INVENTION

It is known that animals, for example equine animals, such as horses, ponies and the like can on occasion suffer from wounds and skin disorders. Typically, wounds are treated by washing out the wound with saline solution or disinfectant and, if appropriate, dried and then covered with a wound dressing. The wound dressing is then secured in place with a bandage. Often, although not exclusively, the bandage used is a disposable, “one-use” type of bandage that is discarded after a single use. These bandages are cohesive and are typically manufactured from a combination of high strength elastic, polyester, non-woven cloth and a latex coating. Examples of such types of bandages are those manufactured by 3M Corporation and marketed under the trade name “Vetrap™”. Re-use of this type of bandages is not recommended due to the risk of spread of infection. Furthermore, this type of bandage is not washable.

The type of wound dressings used to treat wounds suffered by animals, e.g. equine animals, differ depending on the situation and severity of the wound. Typically, the wound dressings are also “one-use” dressings which are discarded immediately after use. Due to the size of area often to be treated on an equine animal, the wound dressings have to be of a certain size and are often expensive. Furthermore, the cost may be high since several dressings may be required to treat a wound during the course of the wound healing process.

Equine animals often suffer from skin disorders including a disorder known colloquially as “mud fever”. Mud fever typically affects the lower limbs of the equine and causes irritation and dermatitis. It can range from mild skin irritations to very painful skin sores with underlying infection. It is often caused by a mixture of bacteria e.g. Dermatophilus congolensis and Staphylococcus spp. Usually, the equine animal's skin acts as a protective barrier. However, the integrity of the epidermis can become compromised and allow bacteria to enter and cause infection. Mud fever can also be caused by fungal organisms. Other commonly used terms for “mud-fever” include pastern dermatitis, grease heel and greasy heel.

The symptoms of mud-fever include scabbing; small, circular ulcerated moist lesions beneath scabs, purulent discharge from scabs, deep fissures in the skin and hair loss. In addition, mud-fever may cause lameness, heat, swelling and pain. If the equine animal is severely affected, it may suffer from lethargy, depression and loss of appetite together with secondary lymphangitis.

It is not known what exactly causes mud fever, however, as the name suggests, it seems to be exacerbated by muddy wet conditions. Mud fever seems to be more prevalent in the winter months but it is not limited to these months and therefore it is believed that other factors contribute to the condition and prevent its resolution. Other factors which may lead to the condition include contact allergies, rubbing boots and the like.

There are a number of treatments available for mud-fever. Typically, the affected limb(s) is cleaned using warm water and then dilute antiseptic solution. The scabs are removed and a wound gel or ointment is applied to the affected area. Other substances which have been touted as treating mud fever include zinc, castor oil and lead acetate. If the infection is severe and swelling occurs, intravenous antibiotics and anti-inflammatories may be administered. In some cases, these treatments are sufficient to heal the affected area. However, treatment can take several weeks to work, if at all, and affected equines are prone to re-infection.

Furthermore, there are limited ways available to prevent mud-fever occurring in the first place or re-occurring following an initial successful treatment. One way is the use of so-called “turn-out” boots which are placed on one or more limbs of the equine before the equine is turned out into a field. These turn-out boots are typically made from a material such as neoprene and are designed to prevent contact with the wet muddy conditions that can cause or exacerbate mud-fever. However, it is not possible to keep these boots fully dry in inclement weather and therefore the skin underneath the boots eventually becomes wet, which may cause mud-fever to occur or aggravate already affected areas of the skin. In addition, mud can become attached to the turn out boots, making it a messy and difficult job to remove them from the limbs of the equine animal when it comes in from the field into a stable or shelter. The appearance of mud-fever is therefore often a regular occurrence, particularly when weather conditions change and the equine animal is subjected to muddy wet conditions.

Regardless of whether an animal is suffering from a skin disorder or wound on one or more of its limbs, it is sometimes advantageous to cover or protect portions of the animal's limbs for a short or prolonged period of time. This may be to prevent knocks to the animal's limbs e.g. whilst stabled, or to keep the animal warm or to provide support. Typically, thick woven bandages are used for this purpose. It is important that bandaging is done correctly and the pressure the bandage applies is both even and suitable for purpose. That is to say, the bandage must be applied with a pressure that is sufficient to keep the bandage in place over a prolonged period of time but not too great so as to reduce blood supply to the limb underneath. Incorrectly applied bandages can have a serious effect on the limb beneath, with uneven, overly tight bandages causing injury to the underlying tissues such as tendons. Bandages can also be difficult to apply especially if the animal is restless or agitated.

To avoid these disadvantages, padded “boots” or “wraps” can be used which can be quickly applied to the limb and then secured with securing means, often in the form of a hook and loop mechanism, that are attached to the boot or wrap. The padded boots or wraps currently available suffer from the disadvantage that they are not shaped to conform closely to the limb to which they are applied. As a result, the boots often slip down the limb, leaving the limb area which is to be protected without protection. Furthermore, the equine animal can tread on the boot or wrap causing the animal to stumble and potentially injury itself. This is especially problematic for “flight” animals such as horses and ponies which are prone to panic if they feel restricted or something unexpected happens.

It is therefore apparent that there is a need for improved products and methods for the treatment and prevention of mud-fever and other skin disorders, particularly those affecting equine animals. It is also apparent that there is a need for improved products to protect the limbs of equine animals from knocks and injury.

### SUMMARY OF THE INVENTION

It is an aim of certain embodiments of the present invention to at least partly mitigate the above-mentioned problems.

In one aspect of the present invention, there is provided an apparatus for covering a target region of an animal comprising a multi-layer cover body comprising an inner layer, a first intermediate layer and an outer layer, each layer being laminated to at least one adjacent layer of the multi-layer body.

In one embodiment, the animal is an equine animal.

In one embodiment, the inner layer comprises a target region contact layer, the first intermediate layer comprises a cushioning layer and the outer layer comprises an impact resistant layer. In one embodiment, the apparatus further comprises a layer which comprises an antibacterial substance.

In one embodiment, the apparatus comprises an activated carbon cloth layer between the inner layer and the first intermediate layer. In an embodiment, the activated carbon cloth layer further comprises the anti-bacterial substance. Aptly, the activated carbon cloth layer is impregnated with metallic silver or a silver salt.

In one embodiment, the cover body comprises no seams. Aptly, the interior of the cover body does not comprise seams. In one embodiment, the apparatus further comprises a peripheral region around an outer edge of the cover body which is bound by a plurality of stitches.

In one embodiment, the apparatus further comprises at least one securing strap locatable to releasably secure the cover body at the target region. Aptly, the at least one securing strap is detachable from the cover body. Aptly, the at least one securing strap comprises at least one end region which comprises a releasable securing member.

In one embodiment, the at least one securing strap comprises a further end region which comprises a releasable securing member. In one embodiment, the at least one securing strap is elasticated.

In one embodiment, each strap is secured at a first end region thereof to the cover body and further comprises a second end region, wherein at least one of the first end region and the second end region of the strap comprises a releasable securing member.

In one embodiment, the apparatus further comprises a cover body which has a central substantially rectangular body region and a first and second substantially semi-circular end region extending from imaginary minor sides of the central region.

In one embodiment, the target region comprises a heel region of the equine animal and the apparatus comprises a heel boot.

In one embodiment, the apparatus comprises a cover body which has a central substantially rectangular body region and a first and second pair of substantially semi-circular ears extending from imaginary major sides of the central region. Aptly, the cover body comprises at least one cut-out region between ears on each respective side of the cover body.

In one embodiment, the target region is a hock. Aptly, the apparatus comprises a hock boot.

In one embodiment, the target region is a region of the equine animal surrounding a knee. Aptly, the apparatus is a knee boot.

In one embodiment, the apparatus comprises a cover body which has a central substantially rectangular body region and a first and second substantially semi-circular ear extending in an opposed configuration at a first end thereof, and a first and second substantially rectangular flap extending in an opposed configuration at both a central and further end region thereof. Aptly, the cover body comprises at least one cut-out region between an ear and flap on each respective side of the cover body.

In one embodiment, the target region further comprises a region of the equine animal surrounding a cannon bone. Aptly, the apparatus comprises a stable boot.

In a second aspect of the present invention, there is provided a kit comprising an apparatus for covering a target region of an animal comprising a multi-layer cover body comprising an inner layer, a first intermediate layer and an outer layer, each layer being laminated to at least one adjacent layer of the multi-layer body. Aptly, the kit comprises an apparatus as described herein.

In one embodiment, the animal is an equine animal.

Aptly, the kit further comprises at least one securing strap locatable to releasably secure the cover body at the target region, wherein the at least one securing strap is detachable from the cover body and comprises at least one end region which comprises a releasable securing member. In one embodiment, the kit comprises at least two securing straps, optionally the kit comprises at least three securing straps and further optionally the kit comprises at least four securing straps. Aptly, the at least one securing strap comprises a further end region which comprises a releasable securing member.

In one embodiment, the kit comprises more than one apparatus as described herein.

In a third aspect of the present invention, there is provided a method of treating a wound of a target region of an animal comprising securing the apparatus described herein to the target region of the equine animal. In one embodiment, the animal is an equine animal.

In a fourth aspect of the present invention, there is provided a method of treating or preventing a skin disorder of a target region of an animal comprising securing the apparatus as described herein to the target region of the equine animal. Aptly, the skin disorder is mud-fever. In one embodiment, the animal is an equine animal.

In a fifth aspect of the present invention, there is provided a method of reducing or preventing swelling of a target region of an equine animal comprising securing the apparatus as described herein to the target region of the equine animal.

In a sixth aspect of the present invention, there is provided an activated carbon cloth for use in the reduction or prevention of swelling of a limb. Aptly, the activated carbon cloth is comprised with an apparatus as described herein. Aptly, the activated carbon cloth is for use to reduce or prevent swelling in the limb of an equine animal.

In one embodiment, the apparatus as described herein is for use in the treatment of an infection caused by D. congolensi. In one embodiment, the apparatus is for use in the treatment of an infection caused by P. aeroginosa. Aptly, the infection is suffered by an equine animal.

In one embodiment, the activated carbon cloth is for use in the treatment of an infection caused by D. congolensi. In one embodiment, the activated carbon cloth is for use in the treatment of an infection caused by P. aeroginosa. Aptly, the infection is suffered by an equine animal.

Embodiments of the present invention are for use on equine animals. As used herein, the term “equine animals” includes for example horses, ponies, donkeys, mules and the like.

Whilst the apparatus of the invention is described herein with reference to equine animals, it will be understood that the apparatus may be for use to cover a target region of a non-equine animal, for example, humans, cats, dogs and/or cattle.

### BRIEF DESCRIPTION OF THE FIGURES

Embodiments of the present invention will now be described hereinafter by way of example only, with reference to the accompanying drawings in which:

FIG. 1 illustrates a laminate structure of an embodiment of the present invention;

FIG. 2 illustrates an embodiment of the present invention. FIG. 2A and FIG. 2B are representations of an apparatus adapted to fit around the upper portion of the hoof and lower part of the pastern region of an equine animal. FIG. 2A illustrates the outer surface of a cover body. FIG. 2B illustrates the inner surface of the cover body. FIG. 2C illustrates an inner surface of an embodiment of a securing strap as described herein. FIG. 2D illustrates an outer surface of an embodiment of a securing strap as described herein;

FIG. 3 illustrates the positioning of the embodiment of FIG. 2A when in use;

FIG. 4 illustrates an alternative embodiment of the present invention. In particular, FIG. 4 is a representation of an apparatus adapted to be secured around a hock joint of an animal. FIG. 4A illustrates the apparatus positioned in use, i.e. around the hock joint of the animal. FIG. 4B illustrates an outer surface of a securing strap for use with the cover body shown in FIGS. 4D and 4E. FIG. 4C illustrates an inner surface of the securing strap. FIG. 4D illustrates an outer surface of the cover body whilst FIG. 4E illustrates an inner surface of the cover body;

FIG. 5 illustrates an alternative embodiment of the present invention. In particular, FIG. 5 is a representation of an apparatus adapted to be secured around the knee joint of an equine animal. FIG. 5A illustrates an outer surface of a cover body of the embodiment and FIG. 5B illustrates an inner surface of the cover body. FIG. 5C illustrates an outer surface of an embodiment of a securing strap for use with the boot body and FIG. 5D illustrates an inner surface of the securing strap;

FIG. 6 illustrates an alternative embodiment of the present invention. In particular, FIG. 6 is a representation of an apparatus adapted to be secured around a cannon bone region of an equine animal. As shown in FIG. 6, the apparatus of the illustrated embodiment extends to cover a pastern region and the upper portion of the equine's hoof. FIG. 6A illustrates an outer surface of the cover body and FIG. 6B illustrates an inner surface of the cover body. FIG. 6C illustrates the cover body in use when secured around the cannon bone and pastern regions of the equine animal.

### DETAILED DESCRIPTION OF EMBODIMENTS OF THE INVENTION

Throughout the description and claims of this specification, the words “comprise” and “contain” and variations of them mean “including but not limited to” and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of the features and/or steps are mutually exclusive. The invention is not restricted to any details of any foregoing embodiments. The invention extends to any novel one, or novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

Embodiments of the present invention relate to products for the protection of animals e.g. equine animals from injury and disease. Particularly, although not exclusively, embodiments of the present invention relate to apparatus to protect the limbs, particularly, the lower portion of the limbs, of equine animals. Furthermore, embodiments of the present invention relate to apparatus which are capable of encouraging wound healing and the prevention of skin disorders.

Embodiments of the present invention are described below:

In a first aspect of the present invention, there is provided an apparatus for covering a target region of an animal comprising a multi-layer cover body comprising an inner layer, a first intermediate layer and an outer layer, each layer being laminated to at least one adjacent layer. In one embodiment, the animal is an equine animal. In one embodiment, the animal is a non-equine animal e.g. a cat, a dog, a cow or a sheep.

Thus, the apparatus comprises a cover body which comprises a laminate structure. In one embodiment, the laminate comprises three layers, wherein each layer is constructed of a different material to the other layers. In one embodiment, the cover body comprises a laminate structure which comprises four layers, wherein each layer is constructed of a different material to the other layers.

In one embodiment, the outer layer comprises a surface facing outwardly from the limb of the equine. The outer layer and details of the material which may be used to construct the outer are described in more detail below.

The cover body may also comprise an inner layer which comprises a target region contact layer. The target region contact layer may comprise a surface which faces inwardly to the limb of the equine animal. The material of the inner layer should be suitable for contact with the skin of the equine animal and therefore should not cause irritation. Further details of suitable material are given below.

The cover body may also comprise a first intermediate layer. The first intermediate layer may comprise a cushioning layer. In one embodiment, the cushioning layer comprises a material which is capable of protecting the limb of the equine animal from knocks and preventing injury to the equine animal.

The cover body may further comprise an additional layer which comprises an anti-bacterial substance or material. In one embodiment, the cover body comprises a layer comprising an activated carbon cloth or fabric. Details of the activated carbon fabric and an example of how it is made are given below. In embodiments in which the cover body comprises a layer of activated carbon fabric, the apparatus may be used to encourage healing of wounds on the limbs of the equine animal which are covered by the cover body of the apparatus. In addition, apparatus which comprise at least one activated carbon layer may be used to prevent or reduce the likelihood of the equine animal developing skin disorders on its limbs or portion of limbs (i.e. target regions) which are covered by the apparatus. In one embodiment, the apparatus may be used to treat skin disorders, for example mud-fever, or prevent the development of such skin disorders. In one embodiment, the apparatus may be used to reduce swelling of an equine's limb or portion thereof. Such swelling may be due to infection or other causes. In one embodiment, the apparatus may be used to prevent or reduce lymphangitis.

In one embodiment, the cover body comprises additional layers to those described above e.g. additional layers of cushioning material or additional layers of antibacterial material. In one embodiment, the laminate structure is such that the cover body is breathable thus enabling moisture from the animal's limb to be wicked through the cover body away from the limb.

In one embodiment, the apparatus comprises an outer layer. The outer layer may comprise an impact resistance layer. Aptly, the impact resistance layer provides an outwardly facing surface, that is to say, a surface which faces outwardly from the limb to which the apparatus is being applied. Aptly, the impact resistance layer is made of a material which is durable and resistant to tears, abrasion and scuffs. As used herein, the terms “durable” and “durability” refer to the propensity of a fabric so characterized to have suitably high grab and tear strength as well as resistance to abrasion for the intended end use of such fabric, and to retain such desirable properties for an appropriate length of time after fabric use has begun.

In one embodiment, the impact resistance layer comprises a material such as for example a nylon yarn. In one embodiment, the material is a nylon blend fabric e.g. a nylon/polyester blend.

One suitable group of materials for the impact resistance layer is the group of materials marketed under the Cordura® brand. In one embodiment, the impact resistance layer material is selected from nylon, polyester, polycotton, vicose and neoprene. The impact resistance layer material may be coated to increase its durability.

In one embodiment, the impact resistance layer is a layer of material which has a weight of between about 85 gsm (3 oz) to about 425 gsm (15 oz). In one embodiment, the impact resistance layer material has a weight of for example 100 gsm, 150 gsm, 200 gsm, 250 gsm, 300 gsm, or 400 gsm. In one embodiment, the impact resistance layer material has a weight of approximately 255 gsm (9 oz).

Aptly, the apparatus further comprises a peripheral region around an outer edge of the cover body which is bound by a plurality of stitches.

In an embodiment, the outer surface of the outer layer may comprise at least one securing element as described in more detail below. In an embodiment, a first securing element may be provided in the form of a securing strap, one end of which is attached to the outer surface of the outer layer and which is capable of securing to a second securing element provided on the outer surface of the outer layer so as to releasably secure the cover body to the target limb. In one embodiment, the second securing element is in the form of one or more areas of material which provides a hook fastening mechanism.

Alternatively, the first securing element is provided separately from the cover body as described below.

In one embodiment, the first intermediate layer comprises a cushioning layer. In one embodiment, the cushioning layer is positioned adjacent to the outer layer and cushions the limb of the animal from knocks, and thus helping to reduce the likelihood of injury. In one embodiment, the cushioning layer is made from a material such as for example foam. In one embodiment, the cushioning layer is a polypropylene foam. Other types of material which could be used in the cushioning layer include for example other types of foam or a felt.

The precise thickness of the cushioning layer is not essential so long as it provides a suitable protective effect whilst being thin enough for the apparatus to be wrapped around the limb of the animal. Thus, in one embodiment, the cushioning layer is from about 3 mm thick to about 30 mm thick. The cushioning layer may be for example 3 mm, 5 mm, 10 mm, 15 mm, 20 mm, 25 mm or 30 mm thick. In one embodiment, the cushioning layer is a layer of foam of approximately 6.5 mm thickness. In this particular embodiment, the foam is approximately 240 gsm in weight.

The cover body also comprises an inner layer which comprises a target region contact layer that contacts the animal during use. Aptly, the target region contact layer is made from a material which is comfortable and non-irritating for the animal to wear for prolonged periods of time. In one embodiment, the target region contact layer is a layer of polyester, e.g. brushed polyester. In alternative embodiments, the target region contact layer is manufactured from other materials e.g. cotton. The inner layer may comprise a material which has a weight of between 50 gsm to 350 gsm e.g. 50 gsm, 60 gsm, 70 gsm, 100 gsm, 150 gsm, 200 gsm, 250 gsm or 300 gsm. In one embodiment, the inner layer comprises a brushed polyester layer which is of a weight of approximately 120 gsm.

In some embodiments of the invention, the cover body further includes a layer which comprises an antibacterial material. The antibacterial layer may be positioned between the first intermediate layer and the outer layer or alternatively between the first intermediate layer and the inner layer.

In one embodiment, the antibacterial material comprises an activated carbon fabric which acts as an antibacterial material. The activated carbon fabric may be of a weight of between about 50 gsm to about 500 gsm, e.g. 50 gsm, 100 gsm, 150 gsm, 200 gsm, 250 gsm, 300 gsm, 350 gsm, 400 gsm, 450 gsm or 500 gsm. In one embodiment, the activated carbon fabric has a weight selected from 110 gsm, 120 gsm, 130 gsm, 160 gsm, 220 gsm and 240 gsm.

In one embodiment, the equine boot apparatus comprises a layer comprising an activated carbon fabric. In one embodiment, the activated carbon fabric is woven. In an alternative embodiment, the activated carbon fabric is non-woven, for example a felt. In an alternative embodiment, the activated carbon fabric is a knitted fabric. The fabric may be elasticated. The activated carbon fabric may vary in thickness in different embodiments. Aptly, the activated carbon fabric is for example from about 0.2.mm in thickness to about 1 mm in thickness. In one embodiment, the activated carbon fabric layer is approximately 0.2 mm in thickness. In one embodiment, the apparatus comprises a layer of activated carbon fabric which has a thickness greater than 0.2 cm, e.g. 0.3, 0.4, 0.5, 0.6, 0.7, 0.8, 0.9 or 1 mm. In one embodiment, the activated carbon fabric has a thickness of between about 0.5 mm and 1 mm.

Activated carbon may also be referred to as activated charcoal, active carbon or black bone. Carbon becomes activated for adsorption by steaming it or heating it in a vacuum. Activated carbon has a very large surface area and pore volume that gives it a very high adsorption capacity. Activated carbon is considered to have utility in wound healing as a result of its ability to remove toxins from wound areas. In particular, the pores contain functional groups which give rise to Van der Waals forces which provides the surface of the activated carbon fabric with a permanent electrostatic charge. It is this electrostatic charge which is believed to be responsible for drawing bacteria away from a wound or skin into the carbon structure. The Van der Waals forces also immobilise and retain the bacteria within the carbon structure.

In addition, ex vivo and in vitro, activated carbon has been shown to filter inflammatory cytokines and chemokines such as IL-8 and TNFα from blood which is believed to aid in wound healing. Activated carbon can also remove odours from wounds.

In one embodiment, the activated carbon fabric is impregnated with an antimicrobial substance. In one embodiment, the activated carbon fabric is impregnated with an anti-microbial metal, e.g. silver and/or copper. In one embodiment, the activated carbon fabric is impregnated with metallic silver. Alternatively, the activated carbon fabric may be impregnated with an anti-microbially active metal derivate, e.g. oxides or ions.

In one embodiment, the activated carbon fabric comprises 0.05% to 5% by weight of the antimicrobial substance. In one embodiment, the cover body comprises a layer of activated carbon fabric which comprises between about 0.05% to about 5% by weight of silver or a salt thereof.

Silver is considered to have anti-bacterial properties which add to the wound healing effects of activated carbon. Additionally, silver is an anti-inflammatory agent and is able to inhibit matrix metalloproteinase activity.

In one embodiment, the activated carbon cloth is a cloth manufactured and sold by Chemviron Carbon Cloth Division. In one embodiment, the cloth is a cloth sold under the trademark “Zorflex®”. The cloth may be either a knitted cloth or a woven cloth. The Zorflex® cloth has a near 100% activated carbon content and a microporous structure.

In an alternative embodiment, the equine boot assembly comprises a layer of material e.g. a dressing sold under the Actisorb™ trade mark and manufactured by Systagenix Wound Management.

In one embodiment, the layers of the multi-layer cover body are bonded together by an adhesive layer positioned between each layer. The adhesive web may be for example a polyamide adhesive web. Alternatively, the adhesive web could be for example adhesive polyester or polypropylene. The adhesive material may have a weight of between about 5 gsm to about 100 gsm e.g. 5 gsm, 10 gsm, 15 gsm, 20 gsm, 25 gsm, 30 gsm, 35 gsm, 40 gsm, 45 gsm or 50 gsm. In one embodiment, each adhesive layer is a polyamide adhesive web of approximately 20 gsm in weight.

In one embodiment, the apparatus of the present invention may further comprise at least one securing element adapted to releasably secure the cover body in place on an animal's limb or portion thereof. A first securing element in the form of a securing strap may be provided. In one embodiment, the first securing element is provided separately from the cover body of the apparatus and may therefore be considered completely detachable from the cover body. Aptly, the first securing element is a securing strap which is removably attachable to a second securing element comprised on the outer surface of the cover body.

Aptly, the securing strap comprises at least one end region which comprises a releasable securing member. In one embodiment, the securing strap comprises a second end region which comprises a further releasable securing member. The releasable securing member(s) may be for example a hook or loop fastening.

In one embodiment, the securing strap may be attached via the first and second end regions to the second securing element. The strap may provide a surface which is capable of fixing the strap to the second securing element. In one embodiment, the strap and the second securing element together provide a hook and loop fastening. The second securing element may comprise one or more releasable securing members which in one embodiment, are one or more areas of hook or loop fastening material positioned on the outer surface of the cover body.

It is standard practice in the equine industry that boots, wraps and bandages are applied to the limbs of animals such that the boots and bandages are secured on the surface facing away i.e. outwardly, from the animal. This is so that the fastenings are not knocked by the opposing limb of the animal which could lead to the boots slipping or falling off. Additionally, there is risk involved in a person bending down to secure the boots in place since the animal may move suddenly and knock the person, potentially causing injury. This is particularly the case if the animal is agitated e.g. in the case of injury. This risk would be increased if the boots are secured on the inside of the boot since the person has to lean down and towards the animal meaning that it is harder to move out of the way if the animal moves, which could lead to serious injury.

In response to this risk, prior art boots for equine purposes are provided with securing elements that are not detachable and are positioned so that they are secured on the outside of the boot. As a result, boots are “left-handed” or “right-handed” and are therefore typically sold in pairs. This can be costly particularly when they are for use to treat injuries on one limb only.

In contrast, embodiments of the present invention provide a detachable securing element which allows a boot to be both “left-handed” and “right-handed” i.e. interchangeable between left and right limbs of the equine. This provides the advantage that apparatus of embodiments of the present invention can be sold in packs of one only, thus reducing cost for the user. In addition, the provision of a detachable securing element e.g. a securing strap enables the end regions of the straps to be secured more precisely where they are needed to aid a better fit to the individual animal. The apparatus may be for use with one or more than one securing straps. The securing straps may be elasticated to enhance the fit of the apparatus around the target region of the animal. The present invention therefore also provides a kit comprising a cover body as described herein and one or more securing straps.

In alternative embodiments, the securing straps are secured at one end region to the outer surface of the cover body and are releasably secured to the second securing element on the outer surface of the cover body.

Aptly, the apparatus of the present invention is shaped so as to allow the animal's joints to flex. Additionally, the apparatus is shaped so as to provide a secure fit around a region of an equine animal's limb and thus prevent the apparatus from slipping. In one embodiment, the apparatus comprises a cut out region which allows a joint region to flex.

In one aspect of the present invention, there is provided a method of manufacturing an apparatus as described herein comprising laminating an inner layer, a first intermediate layer and an outer layer together. The method may also comprise a step of shaping the laminate to form a multi-layer cover body of the apparatus. The step of shaping may be carried out using, for example, a CAD cutter or the like.

The method may further comprise a step of securing a binding portion around the periphery of the multi-layer cover body. The binding portion may be secured by a plurality of stitches.

The method may also comprise a step of securing one or more securing elements to the multilayer cover body.

In one embodiment, the apparatus comprises a layer of activated carbon cloth or fabric. The activated carbon cloth may be impregnated with an anti-bacterial metal e.g. silver. The metal-impregnated activated carbon cloth can be produced through a carbonization and activation process. The process can be one step, two steps, and can be of a continuous batch nature. The carbonization is typically carried out in an oxygen free atmosphere at temperatures of about 350° C. to about 400° C. The activation can be carried out in a steam or CO₂ atmosphere at about 900° C. to 950° C. The starting material can be a viscose rayon or polyacrylonitrile in the form of woven or knitted materials or nonwoven materials such as felts. Prior to carbonization and activation, the raw material is impregnated with a solution of inorganic halides. This can include halides of the metals such as zinc, aluminum, calcium, magnesium, iron (which all have halides with the common, apparently essential Lewis acid characteristics), lead, cobalt and barium. Additionally or alternatively, antimicrobial metals (“metals” as used herein shall include zero-valent metals, as well as metal precursors and derivatives, such as metal oxides and/or metal ions) such as copper and silver can be incorporated in the impregnation to enhance the antimicrobial properties of the final product.

During the carbonization and activation processes, metal salt is converted first into metal oxide and then metal. For example, a silver salt is converted into silver oxide and finally into silver metal. In one embodiment, the metal is substantially generally uniformly dispersed throughout the cloth, or pre-selected regions of the cloth.

As used herein, the term “impregnated” means the metal(s) securely reside in the cloth in any fashion, for example as a coating on fibres, located in interstitial spaces between fibres, embedded into fibres, or otherwise substantially attached and retained by the cloth throughout the intended uses described herein. The process of manufacture can be that described in U.S. Pat. No. 4,529,623, for example, which patent is incorporated herein by reference. The resulting activated carbon cloth has a microporous structure capable of attracting and capturing molecules.

During impregnation, metal salts, such as silver chloride, are uniformly distributed into the cloth. During the impregnation manufacturing process, a metal salt is converted to nano particulate metal. In an example, the metal particles are deposited to extend through the thickness of the cloth. The amount of metal may be for example 0.05 to 5% by weight of metal. In a preferred example, the metal is silver e.g. metallic silver or is a silver salt. In one example the cloth is activated carbon cloth, FM10 (produced by Chemviron Carbon Cloth Division), impregnated with silver 0.3% by weight of metal. The thickness is 0.5 mm, the weight is 120 g/sqm, and the adsorption capacity for ethyl acetate is 35% by weight. In another example, the cloth is activated carbon cloth, FM30K (produced by Chemviron Carbon Cloth Division), impregnated with silver 0.3% by weight of metal. Alternatively, the activated carbon cloth may be FM50K (produced by Chemviron Carbon Cloth Division).

Typically, prior art products which are used for the treatment of wounds and skin disorders such as mudfever in equines are not reusable and have to be disposed of after one use. Embodiments of the present invention provide the advantage of an apparatus for the treatment of wounds and skin disorders which is re-usable. Particularly, the cover body as described herein is washable and therefore can be regularly cleaned and reused. Provided that the apparatus is cleaned using sodium carbonate (washing soda) and not other types of washing powder, the activated carbon fabric retains its anti-bacterial properties. It is therefore possible to machine wash the apparatus cover body at around 40° C.

In one aspect of the present invention there is provided a method of treating wounds comprising applying an apparatus as described herein to a target region wherein the target region is an area surrounding a wound. The wound may be on a limb of an equine animal. In one embodiment, the apparatus is secured onto a region of the limb. The apparatus may be used for several days without having to be removed. In one embodiment, the apparatus which is for use in the treatment of wounds comprises a second intermediate layer which comprises an activated carbon fabric. In one embodiment, the activated carbon fabric comprises an antibacterial substance e.g. metallic silver. As noted above, activated carbon fabric is considered to have a wound healing effect.

In one aspect of the present invention, there is provided an apparatus for use in the treatment of wounds comprising a multi-layer cover body comprising an inner layer, a first intermediate layer and an outer layer, each layer being laminated to at least one adjacent layer of the multi-layer body. Aptly, the apparatus comprises a second intermediate layer which comprises an activated carbon fabric. The activated carbon fabric may be as described herein.

In one aspect of the present invention, there is provided a method of treating or preventing a skin disorder of a target region of an equine animal comprising securing an apparatus as described herein to the target region of the animal. In one embodiment, the apparatus comprises a second intermediate layer which comprises an activated carbon fabric. The activated carbon fabric may further comprise an antibacterial substance such as for example metallic silver.

The apparatus may be used to treat a skin disorder or alternatively it may be used to prevent a skin disorder. In one embodiment, the skin disorder is mud-fever and affects a region of an equine animal's limb.

In one aspect of the present invention, there is provided an apparatus for use in the treatment or prevention of a skin disorder, said apparatus comprising an inner layer, a first intermediate layer and an outer layer, each layer being laminated to at least one adjacent layer of the multi-layer body. The apparatus may comprise a second intermediate layer which comprises an activated carbon fabric. The activated carbon fabric may be as described herein.

In one aspect of the present invention, there is provided a method of reducing swelling of a target region of an equine animal comprising securing an apparatus as described herein to the target region of the animal. In one embodiment, the apparatus comprises a second intermediate layer which comprises an activated carbon fabric. The activated carbon fabric may further comprise an antibacterial substance such as for example metallic silver. The method may comprise reducing swelling in a region of an equine animal's limb e.g. a knee joint region, a hock joint region, a pastern joint region and/or a cannon bone region.

In one aspect of the present invention, there is provided an apparatus for use in the reduction or prevention of swelling of a target region of an equine animal, said apparatus comprising an inner layer, a first intermediate layer and an outer layer, each layer being laminated to at least one adjacent layer of the multi-layer body. The apparatus may comprise a second intermediate layer which comprises an activated carbon fabric. The activated carbon fabric may be as described herein. The apparatus may be for use in reducing swelling in a region of an equine animal's limb e.g. a knee joint region, a hock joint region, a pastern joint region and/or a cannon bone region.

Embodiments of the present invention are illustrated in the Figures and are described without limitation below.

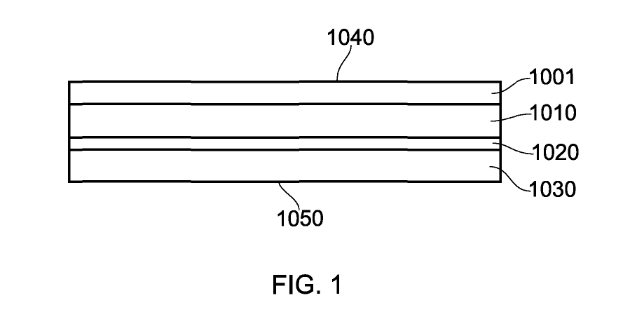

FIG. 1 illustrates a cross-section of a multi-layer cover body of an embodiment of the present invention. As shown in FIG. 1, the cover body includes an outer layer 1001 which includes an impact resistant layer. The outer layer has an outer surface 1040. The cover body also includes a first intermediate layer 1010. In the illustrated embodiment, layer 1010 is a cushioning layer and includes a layer of foam material. The cover body also includes an inner layer which includes a target region contact layer 1030. The target region contact layer provides an inner contact surface 1050. The illustrated embodiment further includes a second intermediate layer 1020 of activated carbon fabric.

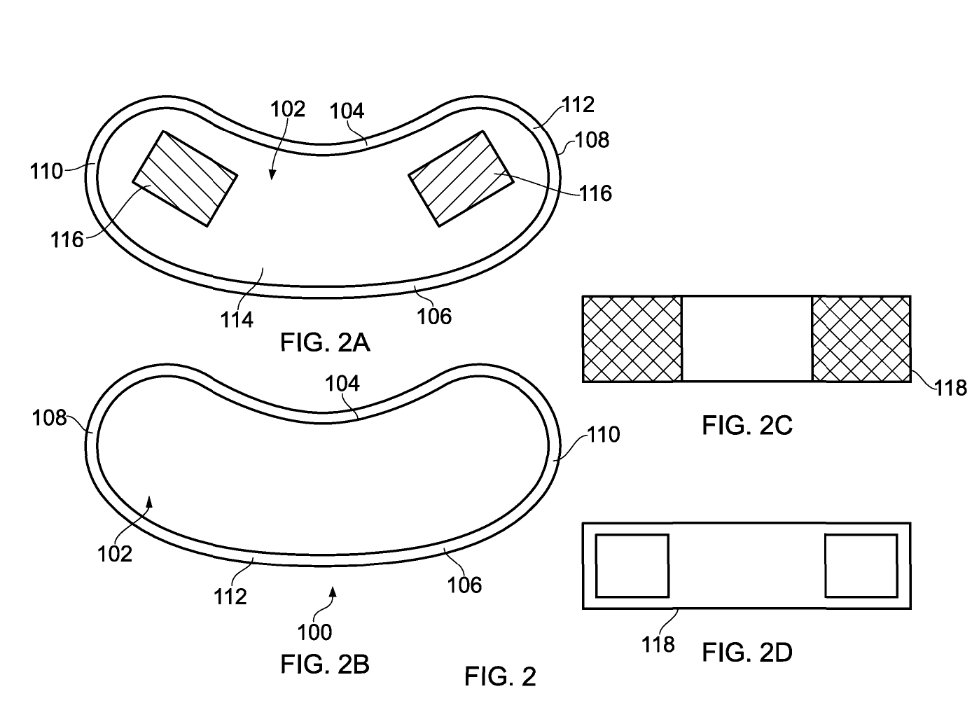

FIG. 2 illustrates an apparatus 100 of an embodiment of the present invention which is adapted to be secured around the pastern and upper hoof region of an equine animal, for example a horse or pony. As shown in FIG. 2A and FIG. 2B, the apparatus includes a cover body 102 which has a roughly kidney bean shape, that is to say, cover body 102 includes an inner arcuate wall 104 and an outer arcuate wall 106 joined by curved end regions 108 and 110. The cover body comprises a strip of binding material 112 secured around the periphery of the cover body 102. The binding material 112 provides a reinforced edge so as to reduce the likelihood of the laminated material of the cover body being damaged during use.

As shown in FIG. 2A, the cover body includes an outwardly facing surface 114 of an outer layer of the cover body. The outwardly facing surface includes securing elements 116 to which end regions of a securing strap 118 are attached during use. The securing strap is illustrated in FIGS. 2C and 2D. The securing elements 116 may be for example hook or loop fastenings e.g. Velcro.

The securing strap 118 may be elasticated so ensure a tight fit around the pastern area and prevent the boot slipping. In the illustrated embodiment, the strap is separate from the boot body and is therefore a detachable securing element. In alternative embodiments (not shown) the strap may be attached e.g. stitched, at an end region to the outer surface of the cover body such that it is not detachable.

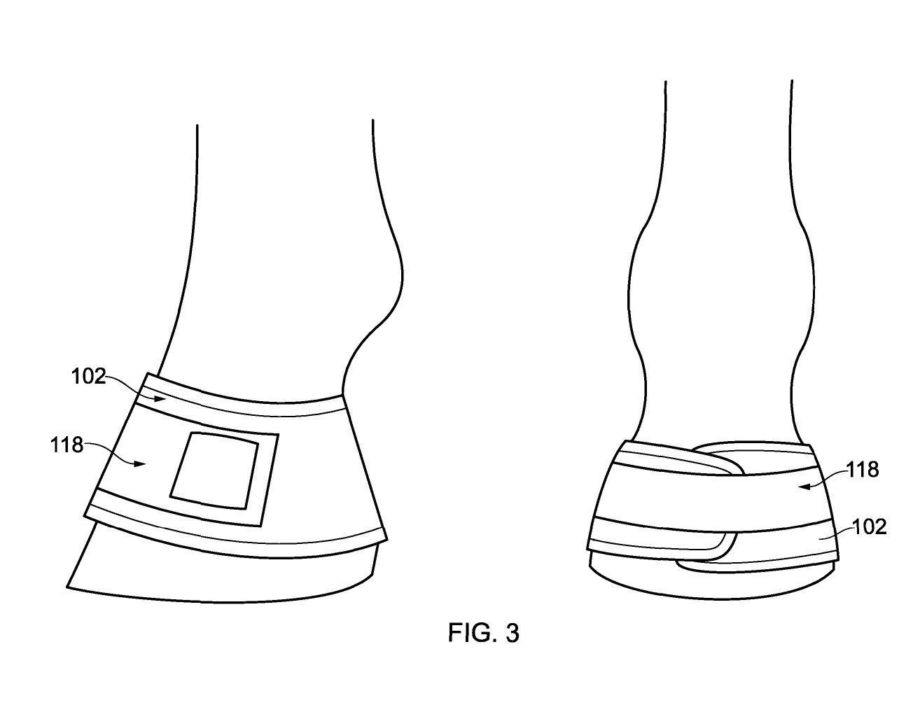

FIG. 3 shows the apparatus of FIG. 2 in place on a horse's limb. Particularly, the apparatus is wrapped around the upper portion of the hoof and secured in place using the strap 118 which releasably fastens to the securing elements 116 of the cover body.

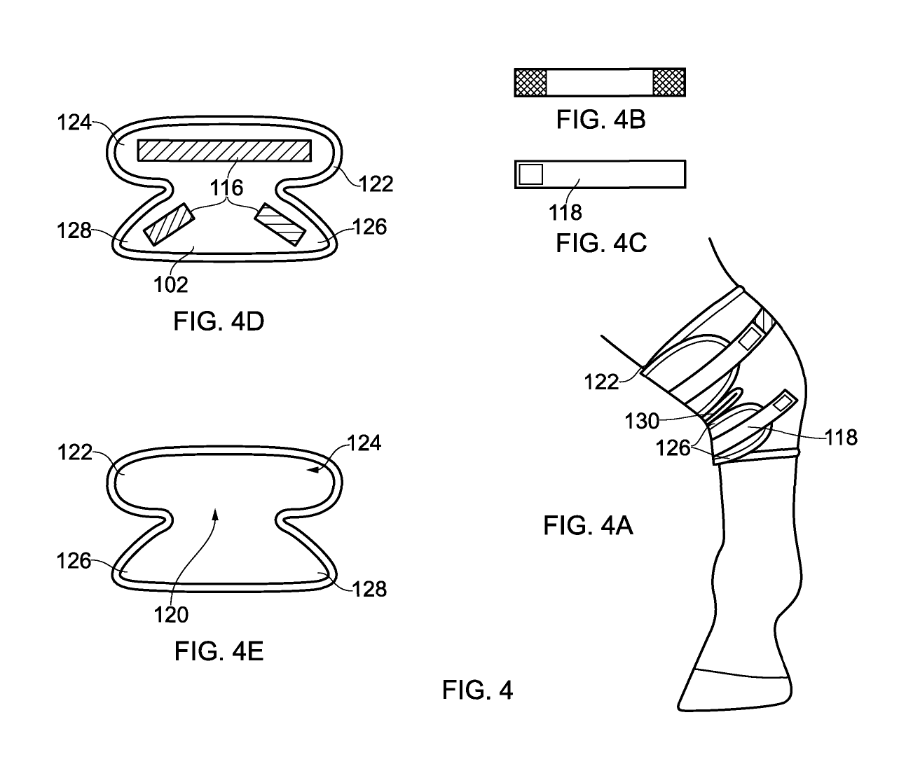

FIG. 4 illustrates an alternative embodiment of the invention which is a hock boot i.e. designed to fit around a hock joint of an animal. The cover body 102 has a roughly rectangular body portion 120 and first pair of ear portions 122, 124 extending from the rectangular body portion. The cover body also includes a second pair of ear portions 126, 128. A cut-out region is provided between the first and second pairs of ear portions. In use, the rectangular body portion 120 contacts the outside of the hock joint as shown in FIG. 4A. The first pair of ear portions wraps around the limb above the joint and the second pair of ear portions wrap around the limb below the joint. The cover body is then secured to the hock region using two securing straps as shown in FIG. 4B and FIG. 4C. The securing straps attach at end regions to securing elements 116 on the outer surface of the cover body. A slit 130 is created between the pairs of ears when secured to the hock region thus allowing the joint to flex when the horse moves. The presence of the slit between the pairs of ears also enables a better fit around the joint.

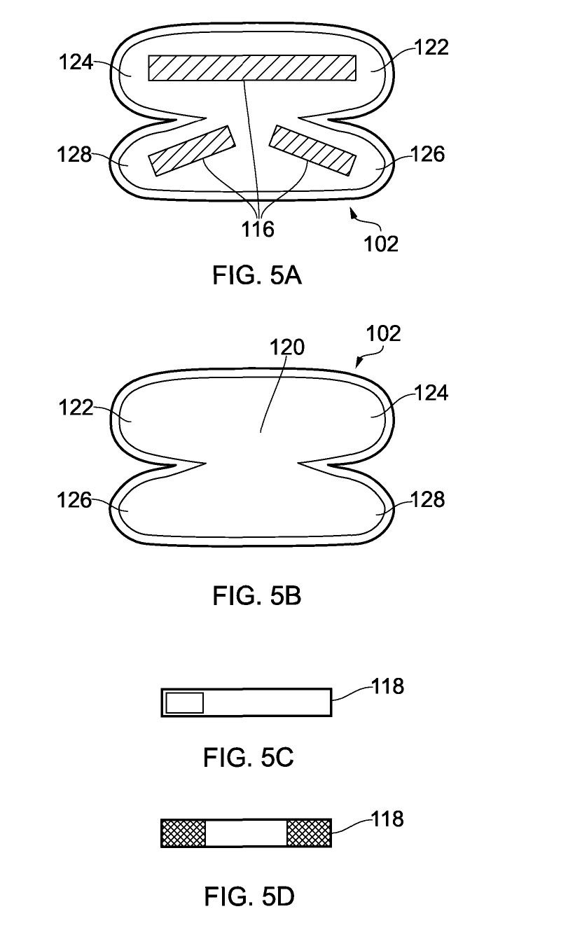

FIG. 5 illustrates a further alternative embodiment of the present invention which is designed to be secured to the region surrounding a knee joint. The cover body 102 comprises a central substantially rectangular body region 120, a first pair of substantially semi-circular ears 122, 124 and a second pair of substantially semi-circular ears 126, 128. A cut-out region is provided between the first and second pairs of ears. During use, the central rectangular body region 120 contacts the front of the knee joint and the first pair of ears are wrapped around the limb above the knee joint and secured in place using a securing strap. The second pair of ears are wrapped around the limb below the knee joint and secured using a second securing strap. Once secured in place, a slit is formed between the first and second pairs of ears allowing the knee joint to bend and flex. This increases the comfort of the horse and reduces the likelihood of the knee boot slipping out of place.

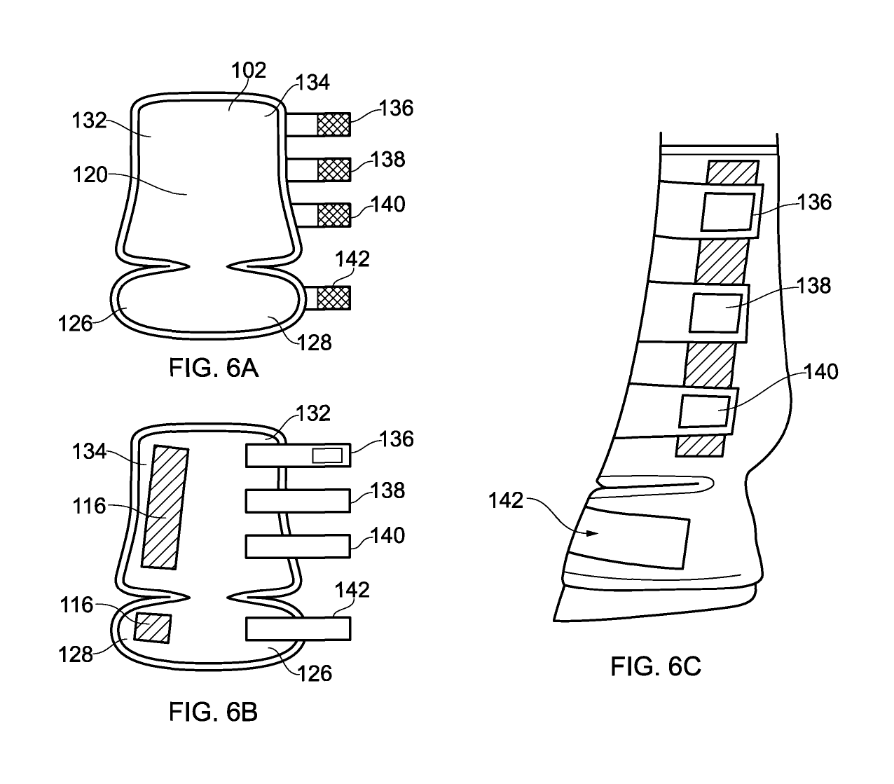

FIG. 6 illustrates a further embodiment which is a stable boot adapted to be secured around a cannon bone region, pastern and fetlock region of a horse. The cover body 102 includes a central substantially rectangular body region 120. A pair of substantially semi-circular ears 126, 128 extends from the central body region. A pair of substantially rectangular flaps 132, 134 extends from the central body region. A cut-out region is provided between the pairs of ears and the pair of rectangular flaps. In the illustrated embodiment, the cover body includes four securing straps 136, 138, 140, 142 which are fixed onto the body portion. In use, the central rectangular body portion contacts the back of the horse's limb i.e. the tendon area and the rectangular flaps are wrapped around the cannon bone region and secured in place. The pair of ears are wrapped around the upper portion of the horse's hoof and secured in place as shown in FIG. 6C.

### EXAMPLES

Antimicrobial testing was carried out by The Department of Biological Sciences at Lincoln University. Zorflex® activated carbon fabric with silver (Zorflex Ag) was tested for effectiveness against P. aeroginosa (a pathogen found in infected animal wounds) and D. congolensis (one of the pathogens known to be responsible for causing equine mudfever).

Testing involved innoculating the fabric (4 cm square patch) with 1 ml of either bacterial suspension, placing them sterile plates (7% sheeps blood agar for D. congolensis and MacConkey agar for P. aeroginosa) and incubating for 2 hours. After 2 hours the patches were then suspended in 50 ml of sterile PBS and washed for 10 mins. The patches were then squeezed out and placed onto sterile plates for later use.

The bacterial concentrations of the washing solutions were determined. This test proved the effectiveness of Zorflex® at adsorbing bacteria. Tests indicated that Zorflex® Ag adsorbed 99% of each bacteria see results below;

| Bacterial Species | Neat Inoculant CFU | Washing liquor | % Decrease |
|---|---|---|---|
| D. congolensis | 1.93 × 10⁵ | 11.0 × 10² | 99 |
| P. aeroginosa | 89.0 × 10⁶ | 18.2 × 10⁴ | 99 |

The squeezed out patches from the above test were then tested for contact inhibition by pressing the patch against sterile plates and then placing the patches on the plate. The plates were then incubated for 48 hrs. Contact inhibition of bacteria growth was observed for both D. Congolensis and P. aeroginosa. This test indicates that once the bacteria are adsorbed by the carbon the growth becomes inhibited.

Finally, two patches of Zorflex® Ag were placed onto inoculated plates (one plate inoculated with D. congolensis and the other with P. aeroginosa). The growth of either bacteria were inhibited underneath the Zorflex® Ag patches. This test indicates that placing Zorflex® Ag over a wound or skin, holding bacteria, the growth rate of bacteria will be inhibited.

### Case Studies

### Case Study 1

Horse 1, an eight year old 16.1 hh Thoroughbred gelding, suffered from a very bad case of mud fever that the owner had tried to treat with barrier creams, homeopathic creams and lastly antibiotic creams without success. Because the mudfever would not clear up, the horse acquired a secondary infection (lymphangitis). After lymphangitis was treated with antibiotics, an activated carbon cloth with silver was used to dress the external wounds. Within three days, the wounds started to close and scab over. After one week, the scabs had turned black and started to crumble off. Within two weeks, the area was scab free and left new pink healthy skin.

Once the mud fever of Horse 1 had healed, the owner used pig oil, a mineral based oil that includes paraffin, as a barrier to the wet conditions. Unfortunately Horse 1 had a major reaction to this oil and all his legs swelled and erupted in sores. After being examined by the vet, the horse was treated with a product containing an activated carbon cloth impregnated with silver to clear up the sores. Within two weeks, the broken skin was healing and the swelling had subsided.

### Case Study 2

Horse 2, a ten year old 16 hh Thoroughbred gelding, suffered from a severe case of mud fever. A number of different treatments were used, including antibiotic creams provided by the veterinary surgeon, however the mud fever had turned in to a large open wound on the back of the hind pastern of the horse. As a result of the open wound, Horse 2 developed secondary infected lymphangitis. Despite being treated every six weeks with antibiotics, the lymphangitis kept re-occurring. The horse was then treated by dressing the wound with a product containing activated carbon impregnated with silver. The dressing was left on for 48 hours before being removed. The improvement was apparent within 48 hours. After a week of dressing using the activated carbon/silver product, the wound was fully healed and the horse could be turned out in the field.

The owner of Horse 2 used the products of the present invention as a preventative measure and the mud fever has not returned.

Sometime after the incidence of mud fever, Horse 2 had an accident in the field which damaged both left and right hind suspensory ligaments causing the fetlock joint to swell. After being taken to the veterinary surgeon for a diagnosis, Horse 2 was put on box rest. A product of the invention as described herein was used on some days, on which days the joints were significantly smaller and the swelling reduced. The joints returned to original (swollen) size if the products were left off for days at a time.

### Case Study 3

Horse 3 is an eighteen year old, 16.3 hh Warmblood gelding, which suffered from re-occurring mud fever over a number of years. Horse 3 was treated as a using products as described herein as a preventative measure to prevent mud fever from re-occurring. Horse 3, as a result of his age and arthritic joints, also suffered from severely swollen legs when stabled. It was noted that following the use of the products as described herein, namely boots which comprised activated carbon impregnated with silver, the horse's joints were not swelling when the boots were on and the horse's movement was much freer when walking out of the stable first thing in the morning. To identify that it is the products as described herein that was contributing to the reduction in swelling, normal stable boots, i.e. boots which did not contain activated carbon impregnated with silver, were placed on the horse's limbs overnight. The result was clear, in that although the joints had not swelled as much as without any boots on, they were significantly larger than with the boots as described herein and the horse's movement was stiffer.

1. Apparatus for covering a target region of an animal comprising:
a multi-layer cover body comprising an inner layer, a first intermediate layer and an outer layer, each layer being laminated to at least one adjacent layer of the multi-layer body, wherein the apparatus further comprises an activated carbon cloth layer between said inner layer and said first intermediate layer.

2. The apparatus as claimed in claim 1, further comprising:
said inner layer comprises a target region contact layer, said first intermediate layer comprises a cushioning layer and said outer layer comprises an impact resistant layer.

3. The apparatus as claimed in claim 1, further comprising a layer which comprises an antibacterial substance.

4. The apparatus as claimed in claim 1, wherein the animal is an equine animal.

5. The apparatus as claimed in claim 3, wherein said activated carbon cloth layer further comprises said anti-bacterial substance, wherein said activated carbon cloth layer is impregnated with metallic silver or a silver salt.

6. (canceled)

7. The apparatus as claimed in claim 1, wherein said cover body comprises no seams.

8. The apparatus as claimed in claim 1, further comprising:
a peripheral region around an outer edge of the cover body which is bound by a plurality of stitches.

9. The apparatus as claimed in claim 1, further comprising:
at least one securing strap locatable to releasably secure the cover body at the target region, wherein the at least one securing strap is detachable from the cover body and comprises at least one end region which comprises a releasable securing member.

10. (canceled)

11. (canceled)

12. (canceled)

13. (canceled)

14. The apparatus as claimed in claim 1, further comprising:
the cover body has a central substantially rectangular body region and a first and second pair of substantially semi-circular ears extending from imaginary major sides of the central region.

15. The apparatus as claimed in claim 14, further comprising:
the cover body comprises at least one cut-out region between ears on each respective side of the cover body.

16. The apparatus as claimed in claim 1, further comprising:
the cover body has a central substantially rectangular body region and a first and second substantially semi-circular ear extending in an opposed configuration at a first end thereof, and a first and second substantially rectangular flap extending in an opposed configuration at both a central and further end region thereof.

17. The apparatus as claimed in claim 16, further comprising:
the cover body comprises at least one cut-out region between an ear and flap on each respective side of the cover body.

18. The apparatus as claimed in claim 1, wherein said target region comprises a heel region of the equine animal and said apparatus comprises a heel boot.

19. The apparatus as claimed in claim 1, wherein said target region is a hock and said apparatus comprises a hock boot.

20. The apparatus as claimed in claim 1, wherein said target region is a region of the equine animal surrounding a knee and said apparatus is a knee boot.

21. The apparatus as claimed in claim 1, wherein said target region further comprises a region of the equine animal surrounding a cannon bone and said apparatus comprises a stable boot.

22. A kit comprising an apparatus for covering a target region of an equine animal comprising:
a multi-layer cover body comprising an inner layer, a first intermediate layer and an outer layer, each layer being laminated to at least one adjacent layer of the multi-layer body.

23. The kit as claimed in claim 22, which further comprises at least one securing strap locatable to releasably secure the cover body at the target region, wherein said at least one securing strap is detachable from the cover body and comprises at least one end region which comprises a releasable securing member.

24. (canceled)

25. (canceled)

26. A method of treating a wound and/or a skin disorder of a target region of an animal comprising:
securing an apparatus for covering a target region of an animal comprising:
a multi-layer cover body comprising an inner layer, a first intermediate layer and an outer layer, each layer being laminated to at least one adjacent layer of the multi-layer body, wherein the apparatus further comprises an activated carbon cloth layer between said inner layer and said first intermediate layer to the target region of the animal.

27. (canceled)

28. (canceled)

29. (canceled)

30. (canceled)

31. A method of reducing swelling of a target region of an equine animal comprising:
securing an
apparatus for covering a target region of an animal comprising:
a multi-layer cover body comprising an inner layer, a first intermediate layer and an outer layer, each layer being laminated to at least one adjacent layer of the multi-layer body, wherein the apparatus further comprises an activated carbon cloth layer between said inner layer and said first intermediate layer to the target region of the equine animal.

32. A method for reducing swelling of a limb of an animal comprising applying an activated carbon cloth to the limb of the animal.

33. (canceled)

34. (canceled)

35. (canceled)

36. (canceled)
